(19) United States
(12) Patent Application Publication
(10) Pub. No.: US 2005/0002886 A1
(43) Pub. Date: Jan. 6, 2005

(54) Dithiols comprising at least one amide functional group and their use in transforming the shape of the hair

(76) Inventors: Michel Philippe, Wissous (FR); Herve Andrean, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/839,233

(22) Filed: May 6, 2004

Related U.S. Application Data
(60) Provisional application No. 60/477,726, filed on Jun. 12, 2003.

(30) Foreign Application Priority Data
May 6, 2003 (FR) 03 05497

(51) Int. Cl. A61K 7/06; A61K 7/09; A61K 7/13

(52) U.S. Cl. 424/70.5

(57) ABSTRACT

An embodiment disclosed herein is the use in cosmetics of at least one dithiol comprising an amide functional group for transforming the shape of the hair. Also disclosed herein are cosmetic compositions comprising at least one dithiol comprising an amide functional group in a cosmetically acceptable medium.

This application claims benefit of U.S. Provisional Application No. 60/477,726, filed Jun. 12, 2003.

Disclosed herein is the use, in cosmetics, of dithiols comprising at least one amide functional group for transforming the shape of hair. Also disclosed herein are cosmetic compositions comprising these dithiols comprising at least one amide functional group in a cosmetically acceptable medium.

Throughout the text, the term “dithiol” encompasses all the possible optical isomers of the dithiol.

As used herein, the term “keratinous fiber” is understood to mean the hair, such as eyelashes, eyebrows, body hairs, hair on the head, and moustaches.

One technique for permanently transforming the shape of hair comprises, in a first step, opening the disulphide bonds of the hair keratin (cystine) using a composition comprising at least one reducing agent (reduction stage), and then, for example, after having rinsed the hair, reconstituting, in a second step, the disulphide bonds by applying at least one oxidizing composition (oxidation stage, also known as setting stage), so as to give the hair the desired shape. This technique may make it possible without distinction to either wave the hair.(permanent waving), or to straighten the hair, it being possible for the hair, as the case may be, to be placed under tension, before, during, or after the reshaping process.

The compositions for carrying out the first step of a permanent wave operation are generally provided in the form of lotions, creams, gels, or powders to be diluted in a liquid vehicle and comprise at least one reducing agent, for example a thiol. Among the thiols, those commonly used are cysteine and thioglycolic acid and its esters, for example glyceryl monothioglycolate. Thioglycolic acid may be effective in reducing the keratin disulphide bonds and may be regarded, at alkaline pH, for example in the form of ammonium thioglycolate, as the reference compound in permanent waving. However, thioglycolic acid may exhibit the disadvantage of giving off an unpleasant smell. A fragrance which allows smells to be masked is generally used for the purpose of overcoming this disadvantage.

Cysteine has a much fainter smell than that of thioglycolic acid but the degree of curling obtained may be much less and may be far from satisfactory. Furthermore, cysteine may require the use of a highly alkaline pH.

Glyceryl monothioglycolate is also highly malodorous. In contrast to the other compounds discussed above, glyceryl monothioglycolate is used at a pH close to neutral, but its performance may be inferior to that of thioglycolic acid.

Various studies have been carried out for the purpose of overcoming the disadvantages of these reducing agents and, to this end, the use of novel reducing compounds or systems has been proposed. Thus, in U.S. Pat. Nos. 2,719,813 and 2,719,814, mention is made of using a mercaptan and its disulphide as a reducing system for the hair, it being recommended for example to adjust the pH of the reducing composition to between 8.5 and 9.5. U.S. Pat. No. 2,719,813 relates to a reducing composition for a permanent wave formed by dithiodiglycerol and thioglycerol, and U.S. Pat. No. 2,719,814 relates to a reducing composition comprising for example thiohydracrylic acid and dithiohydracrylic acid.

Furthermore, U.S. Pat. No. 5,460,806 discloses the use of bis(2-mercaptoethyl) sulphone (BMS) as a reducing agent in a hair process for straightening or curling the hair, without specifying the pH at which reducing solutions comprising such reducing agents are employed. This compound may have very poor solubility in water, and therefore it may be difficult to use the compound under the usual conditions for permanently reshaping the hair.

Finally, the use of certain dithiols in cosmetics is already known. For example, French Patent Application No. 0 204 352, filed by the present assignee, discloses a metal sheathing process which confers, on keratinous fibers, cosmetic properties which may be long-lasting with regard to shampooing. According to this process, a composition comprising at least one metal salt is applied to the keratinous fibers, and then a composition comprising at least one reducing agent is applied to the keratinous fibers. The following dithiols are mentioned among the reducing agents: BMS (bismercaptoethyl sulphone), DTT (dithiothreitol), and DMH (N,N′-dimethyl-N,N′-bis(mercaptoacetyl) hydrazine).

In this French patent application, it is indicated that the keratinous fibers can be reduced prior to applying the composition comprising at least one metal salt. It is possible that this reduction may be carried out with one of the reducing agents described herein, such as the abovementioned dithiols.

Furthermore, the use of the dithiol BMC comprising two amide functional groups for the reduction of proteins is disclosed in WO 98/04734.

Nevertheless, the reducing compositions for permanent waving known to date may still not be entirely satisfactory, given that damage to the hair fiber may be great.

In addition, when a permanent waving composition is applied to hair which has been subjected to dyeing, stripping of the color may be observed and the surface of the hair may be highly damaged. Conversely, if permed hair is dyed, the color obtained may be different from the color normally obtained on natural unpermed hair.

Thus it would be useful to provide a reducing composition for permanently reshaping the hair which is more effective than those which already exist, such as in terms of degree of liveliness or of quality of curling, while reducing the damage to the hair; such a reducing composition would also have minimal effect on the artificial dyeing of keratinous fibers.

To solve this problem, the present inventors provide a method for reducing the disulphide bonds of keratin, by using, in a reducing composition for reshaping a keratinous fiber, for example, at a pH ranging from 6 to 8, dithiols of formula (I) 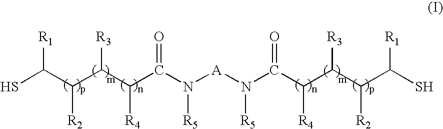
wherein

n, m, and p, which may be identical or different, are chosen from 0 and 1;

R₁, R₂, R₃, and R₄, which may be identical or different, are chosen from hydrogen, COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups;

A is chosen from saturated and unsaturated, linear and nonlinear, C₁ to C₁₈ alkylene groups, which are optionally interrupted by at least one entity chosen from 5-, 6-, and 7-membered aromatic rings; 5-, 6-, and 7-membered nonaromatic rings; heteroatoms; and functional groups such as those listed below

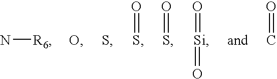

A may also be chosen from 5-, 6-, and 7-membered aromatic and nonaromatic rings.

A is optionally substituted by at least one group chosen from COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups;

R₅ and R₆, which may be identical or different, are chosen from hydrogen, (C₁ to C₄)alkyl groups, (C₁ to C₄)acyl groups, (C₁ to C₄)alkyloxycarbonyl groups, (C₁ to C₄)alkylaminocarbonyl groups, and halogen atoms.

The list and the structures of compounds according to certain embodiments are given below:

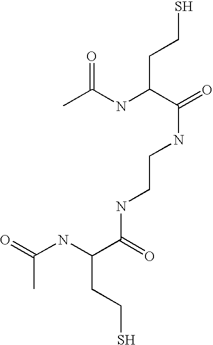

(1) 2-Acetylamino-N-[2-(2-acetylamino-4-mercaptobutyrylamino)ethyl]-4-mercaptobutyramide

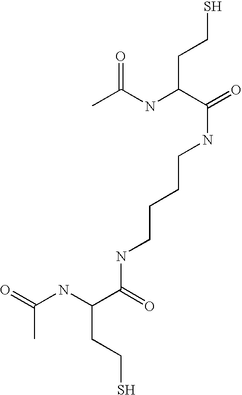

(2) 2-Acetylamino-N-[4-(2-acetylamino-4-mercaptobutyrylamino)butyl]-4-mercaptobutyramide

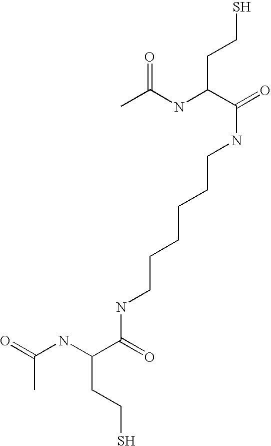

(3) 2-Acetylamino-N-[6-(2-acetylamino-4-mercaptobutyrylamino)hexyl]-4-mercaptobutyramide

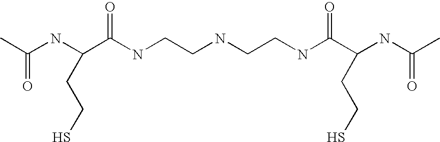

(4) 2-Acetylamino-N-{2-[2-(2-acetylamino-4-mercaptobutyrylamino)ethylamino]ethyl}-4-mercaptobutyramide

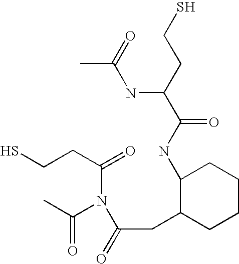

(5) 2-Acetylamino-N-[2-(2-acetylamino-4-mercaptobutyrylamino)cyclohexyl]-4-mercaptobutyramide

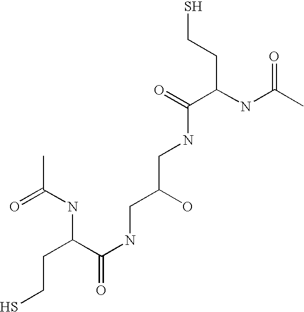

(6) 2-Acetylamino-N-[3-(2-acetylamino-4-mercaptobutyrylamino)-2-hydroxypropyl]-4-mercaptobutyramide

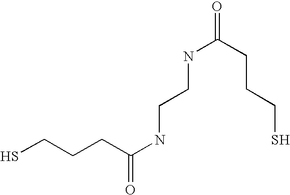

(7) 4-Mercapto-N-[2-(4-mercaptobutyrylamino)ethyl]butyramide.

In certain embodiments, a pH ranging from 6 to 8, such as a pH ranging from 6.5 to 7.5, such as a pH of 7, may be less damaging to the fiber than more basic pH values; thus, the use of a pH in this range may be advantageous compared to the use, at a pH of 8.5 or higher, of thiols such as thioglycolic acid.

Thus, the present disclosure relates, in part, to a process for permanently reshaping the hair by using a reducing composition comprising at least one compound of formula (I).

The compounds of formula (I) may be prepared by reacting, under an inert atmosphere, an amine with a thiolactone at a temperature ranging from 0° C. to the reflux temperature of the solvent for a time ranging from 15 minutes to 12 hours.

Yet another embodiment disclosed herein is a process for preparing a compound of formula (I).

The process as disclosed herein for permanently reshaping the hair comprises applying a reducing composition comprising, as the at least one reducing agent, at least one compound of formula (I). The hair may be shaped by using mechanical means well-known to a person skilled in the art, such as curlers. The reducing composition may be applied before or after optionally shaping the hair, and a setting composition may be applied after the reducing composition, with or without an intermediate or subsequent stage of rinsing or applying an intermediate composition.

In certain embodiments, the process disclosed herein comprises applying the reducing composition disclosed above, comprising, as the at least one reducing agent, at least one compound of formula (I), wherein the hair is shaped by appropriate means; and then applying a setting composition, after an optional rinsing.

As disclosed herein, permanently reshaping the hair may comprise, in a first stage, reducing the disulphide bonds of the keratin by applying, for a time ranging from 5 minutes to 60 minutes, a reducing composition as defined above and then, in a second stage, reforming the bonds by applying an oxidizing composition or optionally by allowing atmospheric oxygen to act as the oxidizing agent.

In certain embodiments, in the case of a permanent wave, a reducing composition as defined above is applied to wet hair wound beforehand onto rollers having a diameter ranging from 4 to 20 mm, the composition optionally being applied as the hair is wound on. The reducing composition is subsequently allowed to act for a time ranging from 5 minutes to 60 minutes, for example from 5 minutes to 30 minutes. Then the hair is copiously rinsed, after which an oxidizing composition which may make it possible to reform the disulphide bonds of the keratin, is applied to the wound hair for an exposure time ranging from 2 to 10 minutes. After the rollers are removed, the hair is copiously rinsed.

The reducing composition may be exposed to heat.

The oxidizing composition may be of the types commonly used and comprises, for example, as the oxidizing agent, at least one agent chosen from hydrogen peroxide, alkaline bromate, persalts, polythionates, and mixtures of alkaline bromate and of persalts. The concentration of hydrogen peroxide may range from 1 to 20 volumes, for example from 1 to 10 volumes. The concentration of alkaline bromate may range from 2% to 12% and that of persalt ranging from 0.1% to 15%, by weight with respect to the total weight of the oxidizing composition. The pH of the oxidizing composition may range from 2 to 10. This oxidation may be carried out immediately or may be delayed.

Permanent reshaping of the hair as disclosed herein, may also comprise a process for straightening the hair, wherein a reducing composition as disclosed herein is applied to the hair and then the hair is subjected to a mechanical deformation which may make it possible to set the hair in its new shape by an operation in which the hair is smoothed with a wide-toothed comb, with the back of a comb, or with the hand. After an exposure time ranging from 5 minutes to 60 minutes, for example from 5 minutes to 30 minutes, the hair is smoothed again. Then the hair is rinsed, an oxidizing or setting composition as defined above is applied to the hair, and the composition is allowed to act for a time ranging from 2 minutes to 10 minutes. Then the hair is copiously rinsed.

In each of the processes described above, it may be possible to insert a stage comprising using a heating iron heated to a temperature ranging from 60° C. to 220° C., for example ranging from 120° C. to 200° C.

In the compositions disclosed herein, the at least one reducing agent of the formula (I) may be present in an amount ranging from 0.05% to 30% by weight, such as from 0.1% to 20% by weight, with respect to the total weight of the reducing composition.

The pH may be obtained using at least one alkaline agent, such as, for example, at least one agent chosen from aqueous ammonia; monoethanolamine; diethanolamine; triethanolamine; 1,3-propanediamine; alkali metals; ammonium carbonate, ammonium bicarbonate; organic carbonates, such as guanidine carbonate; and alkaline hydroxide. The pH may also be obtained by using at least one acidifying agent, such as, for example, hydrochloric acid, acetic acid, lactic acid, oxalic acid, and boric acid.

The reducing composition may also comprise, in combination, another known reducing agent, such as, for example, thioglycolic acid; glyceryl monothioglycolate; glycol monothioglycolate; cysteamine and its C₁-C₄ acylated derivatives, such as N-acetylcysteamine and N-propionylcysteamine; cysteine, N-acetylcysteine; the N-mercaptoalkylamides of sugars, such as N-(2-mercaptoethyl)gluconamide; β-mercaptopropionic acid and its-derivatives; thiolactic acid and its esters, such as glyceryl monothiolactate; thiomalic acid; pantetheine; thioglycerol; alkali metal sulphites; alkali earth metal sulphites; alkali metal bisulphites; alkaline earth metal bisulphites; the N-(mercaptoalkyl)-o-hydroxyalkylamides disclosed in European Patent Application No. EP 354 835; the N-mono- and N,N-dialkylmercapto-4-butyramides disclosed in European Patent Application No. EP 368 763; the aminomercaptoalkylamides disclosed in European Patent Application No. EP 403 267, and the alkylaminomercaptoalkylamides disclosed in European Patent Application No. EP 432 000.

According to one embodiment, the reducing composition also comprises at least one surface-active agent chosen from non-ionic, anionic, cationic, and amphoteric surface-active agents. Mention may be made, among these, of alkyl sulphates, alkylbenzenesulphonates, alkyl ether sulphates, alkylsulphonates, quaternary ammonium salts, alkyl betaines, oxyethylenated-alkylphenols, fatty acid alkanolamides, oxyethylenated fatty acid esters and other hydroxypropyl ether non-ionic surfactants.

When the reducing composition comprises at least one surface-active agent, the at least one surface-active agent may be present at a maximum concentration of 30% by weight, for example a concentration ranging from 0.5% to 10% by weight, with respect to the total weight of the reducing composition.

With the aim of improving the cosmetic properties of the hair or alternatively of lessening or preventing damage thereto, the reducing composition may also comprise at least one cationic, anionic, non-ionic, or amphoteric treating agent.

Mention may be made, among the treating agents that may be used, of those disclosed, for example, in French Patent Nos. 2 598 613 and 2 470 596. Use may also be made, as treating agents, of volatile or non-volatile silicones, linear or cyclic silicones, and mixtures thereof; polydimethylsiloxanes; quaternized polyorganosiloxanes, such as those disclosed in French Patent Application No. 2 535 730; polyorganosiloxanes with aminoalkyl groups modified by alkoxycarbonylalkyl groups, such as those disclosed in U.S. Pat. No. 4,749,732; polyorganosiloxanes, such as the polydimethylsiloxane-polyoxyalkyl copolymers of the dimethicone copolyol type; polydimethylsiloxanes with stearoxy end groups (stearoxy dimethicone); polydimethylsiloxane-dialkylammonium acetate copolymers and polydimethylsiloxane-poly(alkyl betaine) copolymers, disclosed in British Patent No. 2 197 352; polysiloxanes organomodified by mercapto or mercapto-alkyl groups, such as those disclosed in French Patent No.1 530 369 and in European Patent Application No. 295 780; and silanes, such as stearoxytrimethylsilane.

The reducing composition may also comprise at least one additional treating ingredient, such as cationic polymers, for example those used in the compositions disclosed in French Patent Nos. 79.32078 (2 472 382) and 80.26421 (2 495 931); and ionene cationic polymers, such as those used in the compositions disclosed in Luxembourgian Patent No. 83 703; basic amino acids (such as lysine and arginine); acidic amino acids (such as glutamic acid and aspartic acid); peptides and derivatives thereof; protein hydrolysates; waxes; swelling and penetrating agents; and agents which may make it possible to reinforce the effectiveness of the at least one reducing agent, such as mixtures of SiO₂/PDMS (polydimethylsiloxane);dimethylisosorbitol; urea and derivatives thereof; pyrrolidone; N-alkylpyrrolidones; thiamorpholinone; alkyl ethers of alkylene glycol and alkyl ethers of dialkylene glycol, such as, for example, propylene glycol monomethyl ether, dipropylene glycol monomethyl ether, ethylene glycol monoethyl ether, and diethylene glycol monoethyl ether; C₃-C₆ alkanediols, such as, for example, 1,2-propanediol and 1,2-butanediol; 2-imidazolidinone, and other compounds, such as fatty alcohols; lanolin derivatives; active ingredients, such as pantothenic acid; agents for combating hair loss; antidandruff agents; thickeners; suspending agents; sequestering agents; opacifying agents; colorants; and sunscreen agents, as well as fragrances and preservatives.

The reducing composition may be provided in the aqueous form, for example in forms chosen from thickened or non-thickened lotions, creams, and gels.

The reducing composition may also be exothermic, that is to say it gives rise to a degree of warming when applied to the hair, which may be pleasing to the person who is undergoing the first step of the permanent waving or hair straightening process.

The reducing composition disclosed herein may also comprise at least one solvent, such as, for example, ethanol, propanol, isopropanol, and glycerol, at a maximum concentration of 20% by weight, with respect to the total weight of the composition.

The vehicle of the compositions may be chosen from water and aqueous/alcoholic solutions of a lower alcohol, such as ethanol, isopropanol, and butanol.

When the compositions are intended for straightening the hair, the reducing composition may be in the form of a thickened cream, so as to keep the hair as straight as possible. These creams may be prepared in the form of “heavy” emulsions, for example emulsions based on at least one compound chosen from glyceryl stearate, glycol stearate, self-emulsifiable waxes, and fatty alcohols.

It is also possible to use liquids and gels comprising thickening agents, such as carboxyvinyl polymers and copolymers which “stick” the hair together and keep it in the smooth position during the exposure time.

Finally, disclosed herein are cosmetic compositions comprising at least one compound of formula (I) as defined above and at least one compound chosen from non-ionic, anionic, cationic, amphoteric, or zwitterionic surface-active agents and polymers. All these compositions may additionally comprise the additives mentioned above.

Further disclosed herein is a kit, such as for permanently reshaping the hair, comprising, in a first compartment, as a reducing composition, a composition as disclosed herein comprising at least one compound of formula (I) as defined above and optionally comprising at least one compound chosen from non-ionic, anionic, cationic, amphoteric, or zwitterionic surface-active agents and polymers and, in a second compartment, an oxidizing composition.

The invention may be better understood with the help of the following nonlimiting examples.

Other than in the examples, or where otherwise indicated, all numbers expressing quantities of ingredients, reaction conditions, and so forth used in the specification and claims are understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their respective testing measurements.

### EXAMPLES

Reducing compositions comprising a reducing agent of formula (1) to (7), which formula is defined in the description, were prepared.

The compounds (1) to (7) were characterized by proton NMR, mass spectrometry, and quantitative determination of the thiol functional group.

### Synthesis of Compound (1)

A solution of ethylenediamine in dichloromethane (10⁻² mol in 15 ml) was added under an argon atmosphere, with magnetic stirring and at ambient temperature, over 10 minutes to a solution of homocysteine N-acetylthiolactone in dichloromethane (2×10⁻² mol in 35 ml). After two hours, the reaction medium set solid. It was then brought to reflux of the solvent for two hours, was cooled, and the precipitate was filtered off. The precipitate was washed with dichloromethane and pulled dry as much as possible, and was dried on a rotary evaporator. 3.5 grams of odor-free white powder were obtained.

The theoretical weight was 3.79 g, and the yield was 93%.

The other 6 examples were synthesized according to the same procedure.

| Compound | | Homocysteine N- | |
|---|---|---|---|
| number | Amine | acetylthiolactone | Yield % |
| (2) | 1,4-diaminobutane | 6 × 10⁻² mol | 91.3 |
| | 3 × 10⁻² mol | | |
| (3) | 1,6-diaminohexane | 6 × 10⁻² mol | 92.5 |
| | 3 × 10⁻² mol | | |
| (4) | diethylenetriamine | 0.36 mol | 99 |
| | 0.18 mol | | |
| (5) | trans-1,2- | 0.2 mol | 74 |
| | diaminocyclohexane | | |
| | 0.1 mol | | |
| (6) | 1,3-diaminopropanol | 0.1 mol | 93.5 |
| | 5 × 10⁻² mol | | |
| (7) | ethylenediamine | 0.1 mol | 81 |
| | 5 × 10⁻² mol | | |

1. A method for reducing disulphide bonds of keratinous fibers to reshape the keratinous fibers, comprising
applying to the keratinous fibers a reducing composition comprising at least one dithiol of formula (I) 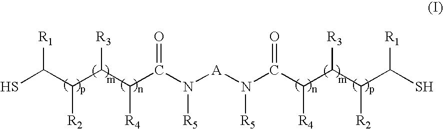
wherein
n, m, and p, which may be identical or different, are chosen from 0 and 1;
R₁, R₂, R₃, and R₄, which may be identical or different, are chosen from hydrogen, COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups;
A is chosen from 5-, 6-, and 7-membered aromatic and nonaromatic rings; and
saturated and unsaturated, linear and nonlinear, C₁ to C₁₈ alkylene groups, optionally interrupted by at least one entity chosen from 5-, 6-, and 7-membered aromatic and nonaromatic rings, heteroatoms, and functional groups;
A is optionally substituted by at least one group chosen from COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups; and
R₅ and R₆, which may be identical or different, are chosen from hydrogen, (C₁ to C₄)alkyl groups, (C₁ to C₄)acyl groups, (C₁ to C₄)alkyloxycarbonyl groups, (C₁ to C₄)alkylaminocarbonyl groups, and halogen atoms.

2. The method according to claim 1, wherein the pH composition has a pH ranging from 6 to 8.

3. The method according to claim 1, wherein A is interrupted by at least one functional group chosen from

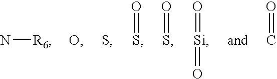

4. The method according to claim 1, wherein the at least one dithiol of formula (I) is chosen from: 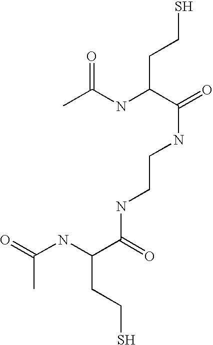
(1) 2-Acetylamino-N-[2-(2-acetylamino-4-mercaptobutyrylamino)ethyl]-4-mercaptobutyramide; 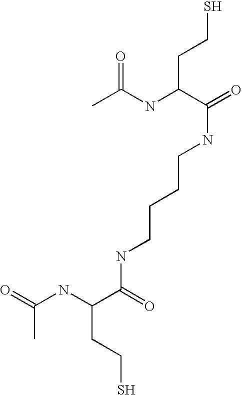
(2) 2-Acetylamino-N-[4-(2-acetylamino-4-mercaptobutyrylamino)butyl]-4-mercaptobutyramide; 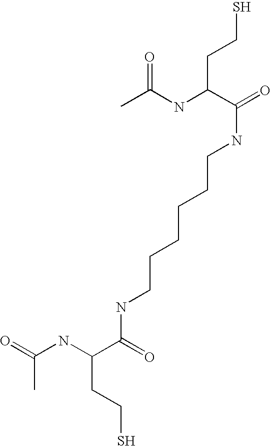
(3) 2-Acetylamino-N-[6-(2-acetylamino-4-mercaptobutyrylamino)hexyl]-4-mercaptobutyramide; 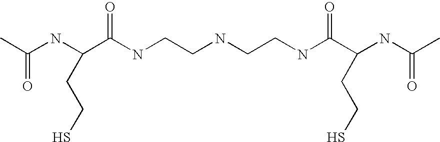
(4) 2-Acetylamino-N-{2-[2-(2-acetylamino-4-mercaptobutyrylamino)ethylamino]ethyl}-4-mercaptobutyramide; 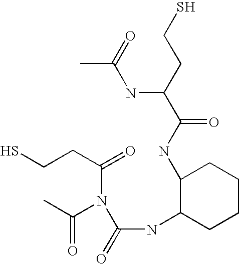
(5) 2-Acetylamino-N-[2-(2-acetylamino-4-mercaptobutyrylamino)cyclohexyl]-4-mercaptobutyramide; 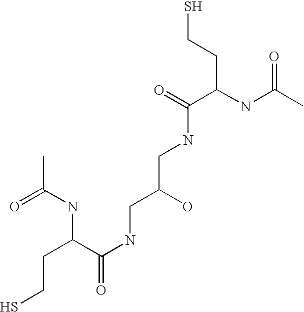
(6) 2-Acetylamino-N-[3-(2-acetylamino-4-mercaptobutyrylamino)-2-hydroxypropyl]-4-mercaptobutyramide; and 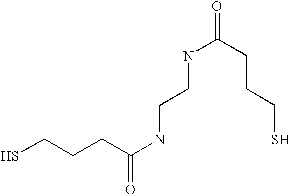
(7) 4-Mercapto-N-[2-(4-mercaptobutyrylamino)ethyl]butyramide.

5. The method according to claim 2, wherein the pH ranges from 6.5 to 7.5.

6. The method according to claim 1, wherein the reshaping is a permanent wave.

7. The method according to claim 1, wherein the keratinous fibers are reshaped by being straightened.

8. A cosmetic composition comprising, in a cosmetically acceptable medium, at least one dithiol of formula (I) 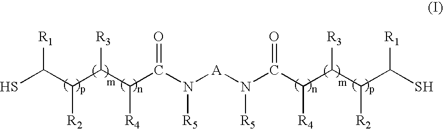
wherein
n, m, and p, which may be identical or different, are chosen from 0 and 1;
R₁, R₂, R₃, and R₄, which may be identical or different, are chosen from hydrogen, COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups;
A is chosen from 5-, 6-, and 7-membered aromatic and nonaromatic rings; and saturated and unsaturated, linear or nonlinear, C₁ to C₁₈ alkylene groups optionally interrupted by at least one entity chosen from 5-, 6-, and 7-membered aromatic and nonaromatic rings, heteroatoms, and functional groups;
A is optionally substituted by at least one group chosen from COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups; and
R₅ and R₆, which may be identical or different, are chosen from hydrogen, (C₁ to C₄)alkyl groups, (C₁ to C₄)acyl groups, (C₁ to C₄)alkyloxycarbonyl groups, (C₁ to C₄)alkylaminocarbonyl groups, and halogen atoms.

9. The cosmetic composition according to claim 8, wherein A is interrupted by at least one functional group chosen from

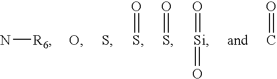

10. The cosmetic composition according to claim 8, wherein the at least one dithiol of formula (I) is chosen from: 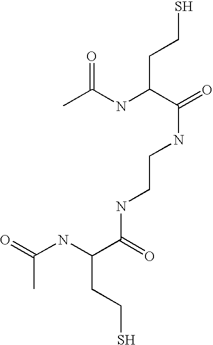
(1) 2-Acetylamino-N-[2-(2-acetylamino-4-mercaptobutyrylamino)ethyl]-4-mercaptobutyramide; 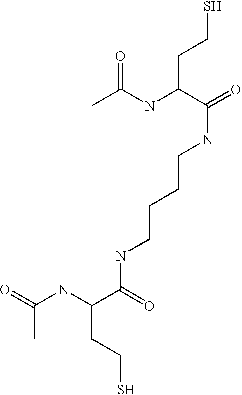
(2) 2-Acetylamino-N-[4-(2-acetylamino-4-mercaptobutyrylamino)butyl]-4-mercaptobutyramide; 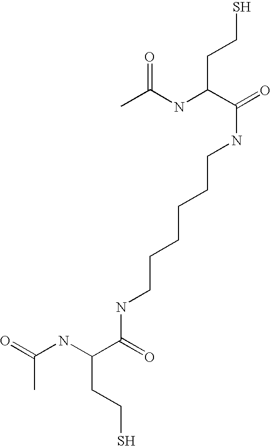
(3) 2-Acetylamino-N-[6-(2-acetylamino-4-mercaptobutyrylamino)hexyl]-4-mercaptobutyramide; 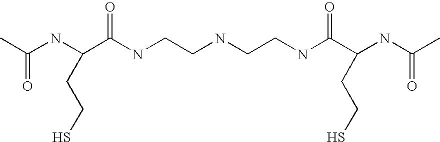
(4) 2-Acetylamino-N-{2-[2-(2-acetylamino-4-mercaptobutyrylamino)ethylamino]ethyl}-4-mercaptobutyramide; 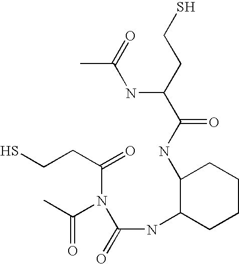
(5) 2-Acetylamino-N-[2-(2-acetylamino-4-mercaptobutyrylamino)cyclohexyl]-4-mercaptobutyramide; 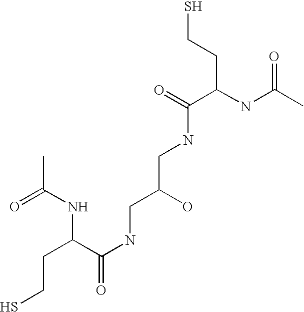
(6) 2-Acetylamino-N-[3-(2-acetylamino-4-mercaptobutyrylamino)-2-hydroxypropyl]-4-mercaptobutyramide; and 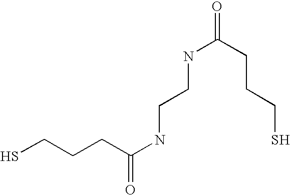
(7) 4-Mercapto-N-[2-(4-mercaptobutyrylamino)ethyl]butyramide.

11. The composition according to claim 8, wherein the at least one dithiol of formula (I) is present in an amount ranging from 0.05% to 30% by weight with respect to the total weight of the reducing composition.

12. The composition according to claim 11, the at least one dithiol of formula (I) is present in an amount ranging from 0.1% to 20% by weight with respect to the total weight of the reducing composition.

13. The composition according to claim 8, further comprising at least one additive chosen from additional reducing agents, nonionic, anionic, cationic, and amphoteric surface-active agents, cationic, anionic, nonionic, and amphoteric treating agents, fatty alcohols, lanolin derivatives, active ingredients, agents for combating hair loss, antidandruff agents, thickeners, suspending agents, sequestering agents, opacifying agents, colorants, sunscreen agents, fragrances, and preservatives.

14. The composition according to claim 13, wherein the active ingredient is pantothenic acid.

15. The cosmetic composition according to claim 8, further comprising at least one compound chosen from nonionic, anionic, cationic, amphoteric, or zwitterionic surface-active agents and nonionic, anionic, cationic, amphoteric, or zwitterionic polymers.

16. A process for permanently shaping hair, comprising
applying to hair at least one reducing composition comprising, as the reducing agent, at least one compound of formula (I) 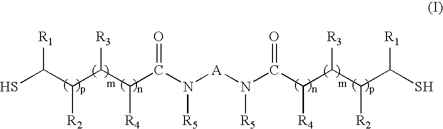
wherein
n, m, and p, which may be identical or different, are chosen from 0 and 1;
R₁, R₂, R₃, and R₄, which may be identical or different, are chosen from hydrogen, COOH, OH, NH₂, (C₁ to C₈)alkyls, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups;
A is chosen from 5-, 6-, and 7-membered aromatic and nonaromatic rings; and saturated and unsaturated, linear and nonlinear, C₁ to C₁₈ alkylenes, optionally interrupted by at least one entity chosen from 5-, 6-, and 7-membered aromatic or nonaromatic rings, heteroatoms, and functional groups;
A is optionally substituted by at least one group chosen from COOH, OH, NH₂, (C₁ to C₈)alkyl groups, (C₁ to C₈)alkylamino groups, (C₁ to C₈)acylamino groups, (C₁ to C₈)acyloxy groups, (C₁ to C₈)alkyloxycarbonylamino groups, (C₁ to C₈)alkylaminocarbonyloxy groups, halogen atoms, and (C₁ to C₈)alkylaminocarbonyl groups; and
R₅ and R₆, which may be identical or different, are chosen from hydrogen, (C₁ to C₄)alkyl groups, (C₁ to C₄)acyl groups, (C₁ to C₄)alkyloxycarbonyl groups, (C₁ to C₄)alkylaminocarbonyl groups, and halogen atoms.

17. The process according to claim 16, wherein A is interrupted by at least one functional group chosen from [chemical structure]
